(19) United States
(12) Patent Application Publication
(10) Pub. No.: US 2021/0268075 A1
(43) Pub. Date: Sep. 2, 2021

(54) PHARMACEUTICAL COMPOSITION AND METHOD OF TREATMENT USING SERRATIOPEPTIDASE, MANNOSE OR ITS DERIVATIVE, AND OPTIONALLY ANTINFECTION AGENTS

(71) Applicant: Nimesh PATEL, Riverside, CA (US)

(72) Inventor: Nimesh PATEL, Riverside, CA (US)

(21) Appl. No.: 17/189,767

(22) Filed: Mar. 2, 2021

Related U.S. Application Data
(60) Provisional application No. 62/984,135, filed on Mar. 2, 2020.

(51) Int. Cl. A61K 38/48; A61K 31/7004; A61K 45/06

(52) U.S. Cl. CPC A61K 38/48; A61K 45/06; A61K 31/7004

(57) ABSTRACT

The present invention relates to method of treating infectious disease, wherein treatment comprises administration of Serratiopeptidase, Mannose or isomers, salts, other derivatives thereof, and one or more antiinfection agents, in same or different compositions to humans or animals. The present invention relates to pharmaceutical composition comprising Serratiopeptidase and Mannose or isomers, salts, other derivatives thereof. The present invention relates to a pharmaceutical composition comprising Serratiopeptidase, Mannose or isomers, salts, other derivatives thereof, and one or more antiinfection agents.

### CROSS-REFERENCE TO RELATED APPLICATION

The present application claims the priority benefit of U.S. provisional application No. 62/984,135, filed Mar. 2, 2020, the entire contents of which is incorporated herein by reference.

### FIELD OF INVENTION

The present invention relates to a method of treating infectious disease, wherein the treatment comprises administration of Serratiopeptidase, Mannose or isomers, salts, other derivatives thereof, and one or more antiinfection agents, in same or different compositions to humans or animals. The present invention relates to a pharmaceutical composition comprising Serratiopeptidase and Mannose or isomers, salts, other derivatives thereof. The present invention relates to a pharmaceutical composition comprising Serratiopeptidase, Mannose or isomers, salts, other derivatives thereof, and one or more antiinfection agents.

### BACKGROUND OF INVENTION

Today, world is facing multiple health challenges. According to WHO's top ten challenges to human health in 2019, five out of 10 challenges are infectious disease. Infections from the different forms of microorganisms are biggest threat to human health. Infectious diseases are caused by pathogenic microorganisms, such as bacteria, viruses, parasites, fungi etc. These infectious diseases spreads directly or indirectly, from one person to another through human or non-human sources. Infectious disease are acute or chronic.

In Infection, the pathogen first invades the host organs and over a period these free floating pathogen attached to the surface of the cell tissue with the help of fimbriae. These pathogen then grows as a colony and secrets extracellular polymers that provides a structural and protective matrix, called Biofilm. This biofilm provides protection to pathogen against an anti-infective agents.

Current therapy in recurrent Infection involves an anti-infective therapy only. The anti-infective agent only eradicates free floating pathogens but the pathogens under the biofilm are protected against the anti-infective agents. These protected pathogens regrow after sometimes and are the source of recurrent infections.

Therefore, there is an urgent need for the improved therapy in the treatment of infectious disease. A solution to current recurrent infection treatment is to dissolve biofilm and block the fimbriae of pathogen so it does not attached to cell surface. This helps to prevent colonization of pathogen and prevents the new biofilm formation.

The present invention provides solution to method of treating infectious disease, wherein treatment comprises administration of Serratiopeptidase, Mannose or its derivatives and one or more antiinfection agents in same or different compositions to humans or animals. This combination keeps the pathogen in free floating state without biofilm or without pathogen attachment and provides improved anti-infection effect on free floating pathogen which helps in eradicating the infection.

### SUMMARY OF THE INVENTION

The present invention relates to a pharmaceutical composition comprising Serratiopeptidase and Mannose or isomers, salts, other derivatives thereof. The pharmaceutical composition optionally may further comprise one or more antiinfection agents. The present invention relates to a method of treating infectious disease, wherein the treatment comprises administration of Serratiopeptidase, Mannose or isomers, salts, other derivatives thereof, and one or more antiinfection agents in same or different compositions to humans or animals.

### BRIEF DESCRIPTION OF THE DRAWINGS

FIG. 1 describes comparative bacterial growth suppression along with the combination as per present invention.

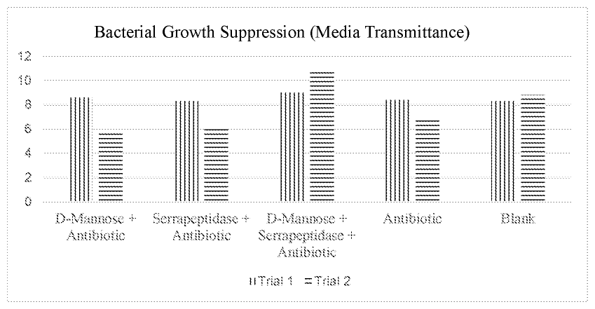

### DETAILED DESCRIPTION OF THE INVENTION

The present invention relates to a pharmaceutical composition and a method of treatment using Serratiopeptidase, Mannose or isomers, salts, other derivatives thereof, and optionally one or more antiinfection agents. The active ingredient as per present invention are used in therapeutically effective amount.

The term “anti-infective agents” or “antiinfection agents” are used interchangeably.

“Therapeutically effective amount” or “effective amount” refers to the amount of a pharmaceutically active agent when administered to a patient, is sufficient to affect such treatment for the disease. The therapeutically effective amount will vary depending on the disease and its severity, and the age, weight, and other conditions of the patient to be treated.

The term “pharmaceutical compositions” herein refers to any composition for administration to human or animal includes but are not limited to immediate release, delayed release, extended release and pulsed-release.

In an embodiment, the present invention relates to a pharmaceutical composition comprising
a. a therapeutically effective amount of Serratiopeptidase, and
b. a therapeutically effective amount of Mannose or isomers, salts, other derivatives thereof.

In a preferred embodiment, the present invention relates to a pharmaceutical composition comprising
a. a therapeutically effective amount of Serratiopeptidase, and
b. a therapeutically effective amount of D-Mannose.

In a preferred embodiment, the present invention relates to an oral pharmaceutical composition comprising
a. Serratiopeptidase in an amount between 0.1 mg and 200 mg, and
b. D-Mannose in an amount between 0.1 mg and 1000 mg.

In an embodiment, the present invention relates to a pharmaceutical composition comprising
a. a therapeutically effective amount of Serratiopeptidase,
b. a therapeutically effective amount of Mannose or isomers, salts, other derivatives thereof, and
c. a therapeutically effective amount of one or more antiinfection agents.

In a preferred embodiment, the present invention relates to a pharmaceutical composition comprising
a. a therapeutically effective amount of Serratiopeptidase,
b. a therapeutically effective amount of D-Mannose, and
c. a therapeutically effective amount of an Antibiotic.

In a preferred embodiment, the present invention relates to an oral pharmaceutical composition comprising
a. Serratiopeptidase in an amount between 0.1 mg and 200 mg,
b. D-Mannose in an amount between 0.1 mg and 1000 mg, and
c. therapeutically effective amount of an Antibiotic.

In an embodiment, the present invention relates to a method of treating infectious disease, wherein said treatment comprises administration of
a. a therapeutically effective amount of Serratiopeptidase,
b. a therapeutically effective amount of Mannose or isomers, salts, other derivatives thereof, and
c. a therapeutically effective amount of one or more antiinfection agents,
   wherein said administration is in same or different compositions and said treatment is administered to humans or animals.

In a preferred embodiment, the present invention relates to a method of treating infectious disease, wherein said treatment comprises administration of
a. a therapeutically effective amount of Serratiopeptidase,
b. a therapeutically effective amount of D-Mannose, and
c. a therapeutically effective amount of an Antibiotic,
   wherein said administration is in same or different compositions and said treatment is administered to humans or animals.

In a preferred embodiment, the present invention relates to a method of treating infectious disease, wherein said treatment comprises administration of
a. Serratiopeptidase in an amount between 0.1 mg and 200 mg,
b. D-Mannose in an amount between 0.1 mg and 1000 mg, and
c. therapeutically effective amount of an Antibiotic,
   wherein said administration is in same or different compositions and said treatment is administered to humans or animals.

In one or more embodiments, a pharmaceutical composition as per present invention includes immediate release, delayed release, extended release or combination thereof.

In one or more embodiments, a pharmaceutical composition as per present invention includes for oral, intravenous, topical, inhalation or other routes of administration.

In one or more embodiments, a pharmaceutical composition as per present invention includes solid, liquid, semisolid, aerosol or other dosage forms.

In one or more embodiments, said pharmaceutical composition or treatment is for urinary tract infection or respiratory tract infection or soft tissue infection or bone infection or skin infection or blood/plasma infection or GI track infection.

In one or more embodiments, a pharmaceutical composition as per present invention comprises one or more antibiotics selected from Aminoglycosides, Carbapenems, Glycopeptides, Quinolones, Penicillins, Fluoroquinolones, Cephalosporins, Sulfonamides, Macrolides, Nitrofurantoin, Metronidazole. Rifamycin, Tetracyclines, Lincomycin, telithromycin and/or other antibiotics.

In a preferred embodiment, invention relates to the treatment of Recurrent Urinary Tract Infection with administration of

a. Nitrofurantoin in an amount between 25 mg and 100 mg,
b. Serratiopeptidase in an amount between 0.1 mg and 200 mg, and
c. D-Mannose in an amount between 0.1 mg and 1000 mg.

In a preferred embodiment, invention relates to the treatment of Recurrent Urinary Tract Infection with administration of

a. Ciprofloxacin/Levofloxacin in an amount between 250 mg and 1000 mg,
b. Serratiopeptidase in an amount between 0.1 mg and 200 mg, and
c. D-Mannose in an amount between 0.1 mg and 1000 mg.

In a preferred embodiment, invention relates to the treatment of Respiratory Tract Infection with administration of

a. Azithromycin/Levofloxacin in an amount between 0.1 mg and 1000 mg,
b. Serratiopeptidase in an amount between 0.1 mg and 200 mg, and
c. D-Mannose in an amount between 0.1 mg and 1000 mg.

In a preferred embodiment, invention relates to a pharmaceutical composition comprising

a. Nitrofurantoin in an amount between 25 mg and 100 mg,
b. Serratiopeptidase in an amount between 0.1 mg and 200 mg, and
c. D-Mannose in an amount between 0.1 mg and 1000 mg.

In a preferred embodiment, invention relates to a pharmaceutical composition comprising

a. Ciprofloxacin/Levofloxacin in an amount between 250 mg and 1000 mg,
b. Serratiopeptidase in an amount between 0.1 mg and 200 mg, and
c. D-Mannose in an amount between 0.1 mg and 1000 mg.

In a preferred embodiment, invention relates to a pharmaceutical composition comprising

a. Azithromycin/Levofloxacin in an amount between 0.1 mg and 1000 mg,
b. Serratiopeptidase in an amount between 0.1 mg and 200 mg, and
c. D-Mannose in an amount between 0.1 mg and 1000 mg.

### Serratiopeptidase

Serratiopeptidase is a proteolytic enzyme prescribed in surgery, orthopaedics, dentistry, otorhinolaryngology and gynaecology for its anti-inflammatory, anti-edemic and analgesic effects. It is produced by non-pathogenic enterobacterium Serratia. This microorganism was originally isolated in the late 1960s from silkworm. Serratiopeptase may be produced by purification from culture of Serratia E-15 bacteria.

Serratiopeptidase is administered in the therapeutically effective amount of between 0.1 mg and 200 mg, preferably between 10 mg and 120 mg. In a preferred embodiment, Serratiopeptidase may be used in the amount of 0.1 mg or more for lung delivery or aerosols. In one or more embodiments of the present invention, Serratiopeptidase may be administered as enteric coated dosage form. Enteric coating consists of pH sensitive polymers which remains intact in the gastric acidic pH (1.5-3.5) and solubilises in the alkaline pH (6.5-7.6) of the small intestines.

### Mannose and its Derivatives

Mannose occurs in microbes, plants and animals. Free mannose is found in small amounts in many fruits and in mammalian plasma. Mannose commonly exists as two different-sized rings, the pyranose (six-membered) form and the furanose (five-membered) form. Each ring closure can have either an alpha or beta configuration at the anomeric position. The chemical rapidly undergoes isomerization among these four forms. D-Mannose can be as α-D-Mannofuranose/α-D-Mannopyranose/β-D-Mannopyranose

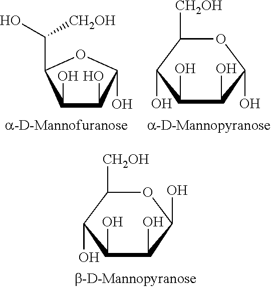

In one or more embodiments, the present invention involves use of preferably D-Mannose. D-Mannose is an epimer of glucose at the C-2 position and exists in nature as a component of mannan. It is a sugar monomer of the aldohexose series of carbohydrates. D-Mannose may be used in the amount of between 0.1 mg and 1000 mg. In a preferred embodiment, D-Mannose can be used in the amount of 0.1 mg or more for lung delivery or aerosols. In a preferred embodiment, D-Mannose can be used in the amount of 10 mg to 1000 mg.

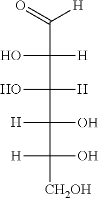 [SMILES: O=CC(O)C(O)C(O)C(O)CO]

### Antiinfection Agents

Infectious diseases are disorders caused by organisms such as bacteria, viruses, fungi or parasites. Microorganisms that cause disease are called pathogens. Pathogens cause disease either by disrupting the bodies normal processes and/or stimulating the immune system to produce a defensive response, resulting in high fever, inflammation and other symptoms. Infectious diseases are transmit in one or more of following

1. From person to person,
2. By insects or other animals,
3. By consuming contaminated food or water.

Anti-infective agents are chemicals which are used to treat infection. Use of anti-infective agents depends on the type of organism targeted. These anti-infective agents include antibacterial (antibiotics), antiviral, antifungal and antiparasitic agents and administered orally, intravenously or by other suitable routes depending on the severity, location and the type of infection.

### Antibiotics

Bacteria are single-celled microorganisms and comes in many shapes including ball-, rod- and spiral-shaped. Infectious bacteria can grow, divide and spread in the body, leading to infectious disease. Many infectious bacteria secretes toxins which increases severity of some diseases.

Antibiotics are medications that kills or inhibits down the growth of bacteria and are widely used in the treatment and prevention of such infections. The different types of antibiotics are Aminoglycosides, Penicillins, Cephalosporins, Carbapenems, Glycopeptides, Quinolones, Fluoroquinolones, Sulfonamides, Macrolides, Nitrofurantoin, Metronidazole, Rifamycin, Tetracyclines, Lincomycin, Telithromycin and/or other antibiotics.

### Antivirals

Viruses are tiny capsules that contain genetic material and replicate only in the living cells of other organisms. They invade cells, multiplies and damage the cells. They can infect human, animals, plants, bacteria and other forms of living bodies.

Antiviral drugs are a class of medication used specifically for treating viral infections. Most antivirals are used for specific viral infections but broad spectrum antivirals are effective against a wide range of viruses. The different types of antivirals are Protease inhibitor, Integrase inhibitor, Reverse transcriptase inhibitor, Neuroamidase inhibitors, Guanosine analogs and and/or other antiviral.

### Pharmaceutical Composition

The pharmaceutical compositions are the different type of medicinal preparation designed for the administration of targeted one or more drugs. The pharmaceutical compositions as per present invention includes immediate release, delayed release, extended release and pulsed-release. The pharmaceutical compositions can be prepared using uniform mixture of two or more drugs. In one or more embodiments, pharmaceutical composition can be prepared with one or more drugs in separate compartment within a single dosage form. The pharmaceutical compositions as per present invention can be administered by oral, topical, inhalation, intravenous or other routes of drug administration. The pharmaceutical compositions as per present invention can be solid, liquid, semisolid, aerosol or any other dosage form. The pharmaceutical compositions as per present invention can be prepared as one or more drug in modified release and other drugs as immediate release in single dosage form.

The oral pharmaceutical dosage form are tablets, capsules, solutions, emulsions, suspensions, syrups, elixirs, aerosol, powders and granules for reconstitution, lozenges, dispersible powders and granules, medicated gums, chewing tablets, effervescent tablets, multi-particulate dosage forms and the likes. The multicompartment dosage form are bilayer tablets, capsule-in-capsule, tablet-in capsule and any other dosage form. The pharmaceutical compositions can be formulated by any techniques known to or appreciated by a person skilled in the art

In an embodiment, the oral pharmaceutical composition further includes optionally any one or a combination of one or more pharmaceutically acceptable excipients, such as but not limited to carriers, diluents, fillers, disintegrants, lubricating agents, binders, colorants, pigments, stabilizers, preservatives, antioxidants and solubility enhancers.

Having described the invention with reference to the different embodiments of the invention, other embodiments will become apparent to one skilled in the art from consideration of the specifications.

The innovation is further defined by reference to the following examples. It will be apparent to those skilled in the art that many modifications, both to the composition and treatment, may be practiced without departing from the scope of this invention.

### Examples

The following In Vitro Testing Demonstrating Suppression of Bacterial Growth Is Superior With Serratiopeptidase, D-Mannose and Antibiotic compare to Serratiopeptidase and Antibiotic, D-Mannose and Antibiotic and Antibiotic Alone were performed.

A nutrient media was prepared with a plastic thread suspended in it. The nutrient media was inoculated with bacteria and was kept overnight leading to the formation of biofilm. On next day, plastic thread was moved to new nutrient media with either D-Mannose or Serrapeptidase or both D-Mannose and Serrapeptidase or Blank (No Addition) and bacterial was allowed grows for 6 to 7 hours. After 6-7 hours, plastic thread was added to new nutrient media and then antibiotic was added.

### Material Preparations:

### 1) E. Coli Culture 10×10⁶ CFU/ml Preparation:

1 ml of Stock E. Coli ATCC 8739 (Culture Count 10×10⁸ CFU/ml) was diluted to 10 ml with buffered peptone water with NaCl. The 1 ml of resulting culture (10×10⁷ CFU/ml) was diluted to 10 ml with buffered peptone water with NaCl to make E. Coli Culture (10×10⁶ CFU/ml)

2) Buffered Peptone water with NaCl Preparation:

16 Gm of peptone was suspended in 1000 ml Distilled water. Heat it if necessary to dissolve the media. To this solution, 5 gm of NaCl and 3.5 gm of Disodium Phosphate was added. The solution was sterilized by autoclaved at 15 lbs pressure at 121° C. for 15 Minutes.

### 3) Nutrient Liquid Broth (Soybean Casein Digest Medium) Preparation:

30 gm of media was suspended in 1000 ml of distilled water. Heat it if necessary to dissolve. The broth was sterilized by autoclave at 15 lbs pressure at 121° C. for 15 minutes.

4) Nutrient Liquid Broth with Glucose (Soybean Casein Digest Medium with Glucose) Preparation:

30 gm of media and 10 gm of glucose was suspended in 1000 ml of distilled water. Heat it if necessary to dissolve. The broth was sterilized by autoclave at 15 lbs pressure at 121° C. for 15 minutes.

### 5) D-Mannose Solution Preparation: Solution F.

500 mg of D-Mannose was dissolved in 10 ml of Distilled Water.

### 6) Serratiopeptidase Solution Preparation: Solution P.

500 mg of Serratiopeptidase was dissolved in 10 ml of Distilled Water.

### 7) Antibiotic (Nitrofurantoin) Preparation: Solution A.

100 mg of Nitrofurantoin Anhydrous was dissolved in 100 ml of Dimethyl Sulfoxide.

### 8) Diluted Antibiotic (Nitrofurantoin) Preparation: Solution A^{dil}.

100 mg of Nitrofurantoin Anhydrous was dissolved in 100 ml of Dimethyl Sulfoxide. 3 ml of resulting solution was diluted to 10 ml with Dimethyl Sulfoxide.

Procedure 1:
1. On first day 6 PM, Five (5) sterile test tubes were added with 50 ml of sterile Soybean Casein Digest Medium. These medium were inoculated with 1 ml of E. Coli culture (10×10 6 CFU/ml @ 6 PM. A thin plastic thread (0.1 mm OD) of the same length were suspended from the middle of all 5 test tubes and keep in overnight (17 hours) at 30° C.-35° C.
2. On second day 11 AM, another Five (5) sterile test tubes were added with 50 ml of sterile Soybean Casein Digest Medium and 500 mg of Glucose.
   a. 1 ml of Solution F (D-mannose solution) was added to test tube 1.
   b. 1 ml of Solution P (Serrapeptidase solution) was added to test tube 2.
   c. 1 ml of Solution F (D-Mannose solution) and 1 ml of Solution P (Serrapeptidase solution) was added to test tube 3.

The thin plastic thread was transferred from old test tube to new test tube on second day 11 AM. All 5 test tubes were kept at 30° C.-35° C. for 7 hours.
3. On second day 6 PM, another Five (5) sterile test tubes were added with 50 ml of Sterile Soybean Casein Digest Medium. Transfer the thin plastic thread from old test tube to new test tube on second day at 6 PM. Keep all 5 test tube at 300-350 C for overnight (17 hours).
4. On third day 11 AM, add 1 ml of Solution A^{dil} (Diluted Antibiotic (nitrofurantoin) solution) to test tube 1, test tube 2, test tube 3 and test tube 4. All 5 test tubes were kept at 30° C.-35° C. for 7 hours.
5. On third day 6 PM, plastic threads were removed from all 5 test tubes. The transmittance of all the 5 test tubes medium was checked at 590 nm.
   a. Test tube 1: With solution F (D-Mannose) and A^{dil} (Diluted Antibiotic): 8.6
   b. Test tube 2: With Solution P (Serrapeptidase) and A^{dil} (Diluted Antibiotic): 8.3
   c. Test tube 3: With Solution F (D-Mannose), P (serrapeptidase) and A^{dil} (Diluted Antibiotic): 9.0
   d. Test tube 4: With A^{dil} (Diluted Antibiotic): 8.4
   e. Test tube 5: Blank: 8.3

Procedure 2:
1. On fourth day 6 PM, Five (5) sterile test tubes were added with 50 ml of sterile Soybean Casein Digest Medium. These test tubes were inoculated with 1 ml of E. Coli culture (10×10⁶ CFU/ml). A thick plastic thread (0.5 mm OD) with same length was suspended from the middle of all 5 test tubes and keep in overnight (17 hours) at 30°-35° C.
2. On fifth day 11 AM, another Five (5) sterile test tubes were added with 50 ml of sterile Soybean Casein Digest Medium with 500 mg of Glucose.
   a. 1 ml of Solution F (D-mannose solution) was added to test tube 1.
   b. 1 ml of Solution P (Serrapeptidase solution) was added to test tube 2.
   c. 1 ml of Solution F (D-Mannose solution) and 1 ml of Solution P (Serrapeptidase solution) was added to test tube 3.

The thick plastic thread was transferred from old test tube to new test tube on fifth day at 11 AM. All 5 test tube were kept at 30°-35° C. for 7 hours.
3. On fifth day 6 PM, another five (5) sterile test tubes were added with 50 ml of Sterile Soybean Casein Digest Medium. Then 1 ml of Solution A (Antibiotic (nitrofurantoin) solution) was added to test tube 1, test tube 2, test tube 3 and test tube 4. The thick plastic thread was transferred from old test tube to new test tube on fifth day at 6 PM. All Five (5) test tube were kept at 30°-35° C. for overnight (17 hours).
4. On sixth day 6 PM, the thread were removed from all 5 test tubes. The transmittance of all the 5 test tubes medium was checked at 590 nm.
   a. Test tube 1: With solution F (D-Mannose) and A (Antibiotic): 5.7
   b. Test tube 2: With Solution P (Serrapeptidase) and A (Antibiotic): 6.0
   c. Test tube 3: With Solution F (D-Mannose), P (serrapeptidase) and A (Antibiotic): 10.7
   d. Test tube 4: With A (Antibiotic): 6.9
   e. Test tube 5: Blank: 8.8

Results—In both test, test tube with D-Mannose, Serrapeptidase and Antibiotic shows the higher transmittance (less bacterial growth) compare to test tube with D-Mannose or test tube with Serrapeptidase or Blank test tube (only Antibiotic).

What is claimed is:

1. A pharmaceutical composition comprising
a therapeutically effective amount of Serratiopeptidase;
a therapeutically effective amount of Mannose or isomers, salts, other derivatives thereof; and
optionally, a therapeutically effective amount of one or more antiinfection agents.

2. The pharmaceutical composition of claim 1, wherein said Serratiopeptidase is in the amount of 0.1 mg to 200 mg.

3. The pharmaceutical composition of claim 1, wherein said Mannose is D-Mannose.

4. The pharmaceutical composition of claim 3, wherein said D-Mannose is in the amount of 0.1 mg and 1000 mg.

5. The pharmaceutical composition of claim 1, wherein said composition comprises a therapeutically effective amount of one or more antiinfection agents.

6. The pharmaceutical composition of claim 5, wherein said antiinfection agents are antibiotics.

7. The pharmaceutical composition of claim 6, wherein said antibiotics are selected from group consisting of Nitrofurantoin, Ciprofloxacin, Levofloxacin and Azithromycin.

8. A method of treating infectious disease, wherein said treatment comprises administration of
a therapeutically effective amount of Serratiopeptidase;
a therapeutically effective amount of Mannose or isomers, salts, other derivatives thereof; and
optionally, a therapeutically effective amount of one or more antiinfection agents.
wherein said administration in same or different compositions and said treatment is administered to humans or animals.

9. The method of treating infectious disease according to claim 8, wherein said Serratiopeptidase is administered in the amount of 0.1 mg to 200 mg.

10. The method of treating infectious disease according to claim 8, wherein said Mannose is D-Mannose.

11. The method of treating infectious disease according to claim 10, wherein said D-Mannose is in the amount of 0.1 mg and 1000 mg.

12. The method of treating infectious disease according to claim 8, wherein said treatment comprises a therapeutically effective amount of one or more antiinfection agents.

13. The method of treating infectious disease according to claim 12, wherein said antiinfection agents are antibiotics.

14. The method of treating infectious disease according to claim 13, wherein said antibiotic is selected from group consisting of Nitrofurantoin, Ciprofloxacin, Levofloxacin and Azithromycin.

15. The method of treating infectious disease according to claim 8, wherein said infectious disease is urinary tract infection.

16. The method of treating infectious disease according to claim 8, wherein said infectious disease is respiratory tract infection.
